(19) United States
(12) Patent Application Publication
(10) Pub. No.: US 2017/0238902 A1
(43) Pub. Date: Aug. 24, 2017

(54) SYSTEM FOR REDUCING A FOOTPRINT OF AN ULTRASOUND TRANSDUCER PROBE

(71) Applicant: General Electric Company, Schenectady, NY (US)

(72) Inventors: Warren Lee, Niskayuna, NY (US); Chester Frank Saj, Amsterdam, NY (US); David Martin Mills, Niskayuna, NY (US); Kwok Pong Chan, Troy, NY (US); Yuji Okumura, Tokyo (JP); Timothy James Fiorillo, Cobleskill, NY (US)

(21) Appl. No.: 15/047,635

(22) Filed: Feb. 19, 2016

(51) Int. Cl. A61B 8/00

(52) U.S. Cl. CPC A61B 8/4444

(57) ABSTRACT

A transducer probe is presented. The transducer probe includes a housing having a probe surface at a first end. Further, the transducer probe includes an acoustic array having an array aperture, wherein the acoustic array is disposed adjacent the probe surface of the housing, and wherein the acoustic array is configured to transmit ultrasound signals towards a target volume. Also, the transducer probe includes a flex interconnect configured to electrically couple the acoustic array to at least one electronic unit. Furthermore, the transducer probe includes an electrical standoff disposed between the acoustic array and the flex interconnect to reduce a footprint of the transducer probe to a first value, wherein the first value is proximate to a lateral size of the array aperture.

### BACKGROUND

Embodiments of the present specification relate generally to an ultrasound transducer probe, and more particularly to a system for reducing a footprint of the ultrasound transducer probe.

Medical diagnostic ultrasound is an imaging modality that employs ultrasound waves to probe acoustic properties of biological tissues and produces corresponding images. Particularly, ultrasound systems are used to provide an accurate visualization of muscles, tendons, and other internal organs to assess their size, structure, movement, and/or pathological conditions using near real-time images. Moreover, owing to the ability to image underlying tissues without use of ionizing radiation, ultrasound systems find extensive use in angiography and prenatal scanning.

Conventional ultrasound systems employ various components, such as a transducer probe that houses an acoustic stack, an interconnect, and an application specific integrated circuit (ASIC). These components are used to transmit and receive ultrasound signals from a target volume in a patient or a subject. However, in these systems, a lateral size of the ASIC and a lateral size of the interconnect are substantially larger than a lateral size of the acoustic stack in the transducer probe. As a result, a footprint of the transducer probe extends beyond the lateral size of the acoustic stack. As will be appreciated, while scanning, transducer probes with smaller footprints are relatively easier to maneuver. By way of example, a transducer probe, such as a transthoracic probe, needs to be positioned in small acoustic windows available between ribs of the patient for cardiac imaging. However, it is difficult to position a conventional transducer probe in these small windows due to the large footprint of the transducer probe.

### BRIEF DESCRIPTION

In accordance with aspects of the present specification, a transducer probe is presented. The transducer probe includes a housing having a probe surface at a first end. Further, the transducer probe includes an acoustic array having an array aperture, wherein the acoustic array is disposed adjacent the probe surface of the housing, and wherein the acoustic array is configured to transmit ultrasound signals towards a target volume. Also, the transducer probe includes a flex interconnect configured to electrically couple the acoustic array to at least one electronic unit. Furthermore, the transducer probe includes an electrical standoff disposed between the acoustic array and the flex interconnect to reduce a footprint of the transducer probe to a first value, wherein the first value is proximate to a lateral size of the array aperture.

In accordance with a further aspect of the present specification, a system for ultrasound imaging is presented. The system includes an acquisition subsystem configured to obtain image data corresponding to a target volume in an object of interest and including an ultrasound probe, wherein the ultrasound probe includes a housing having a first end and a second end, wherein the first end includes a probe surface, and wherein the second end is coupled to a probe cable, an acoustic array having an array aperture, wherein the acoustic array is disposed adjacent the probe surface of the housing, and wherein the acoustic array is configured to transmit ultrasound signals towards a target volume, a flex interconnect configured to electrically couple the acoustic array to at least one electronic unit, and an electrical standoff disposed between the acoustic array and the flex interconnect to reduce a footprint of the transducer probe to a first value, wherein the first value is proximate to a lateral size of the array aperture. Further, the system includes a processing subsystem in operative association with the acquisition subsystem and configured to process the acquired image data to generate one or more images corresponding to the target volume in the object of interest.

In accordance with another aspect of the present specification, a transducer probe is presented. The transducer probe includes a housing having a probe surface at a first end. Further, the transducer probe includes an acoustic array having an array aperture, wherein the acoustic array is disposed adjacent the probe surface of the housing, and wherein the acoustic array is configured to transmit ultrasound signals towards a target volume. Also, the transducer probe includes at least one ASIC configured to electrically couple the acoustic array and configured to receive the ultrasound signals reflected from the target volume. In addition, the transducer probe includes an electrical standoff disposed between the acoustic array and the at least one ASIC to reduce a footprint of the transducer probe to a first value, wherein the first value is proximate to a lateral size of the array.

### DRAWINGS

These and other features, aspects, and advantages of the present disclosure will become better understood when the following detailed description is read with reference to the accompanying drawings in which like characters represent like parts throughout the drawings, wherein:

FIG. 1 illustrates an ultrasound system for imaging a target volume in a subject, in accordance with aspects of the present specification;

FIG. 2 is a diagrammatical representation of a transducer probe having an electrical standoff and a flex interconnect, in accordance with aspects of the present specification;

FIG. 3 is a diagrammatical representation of a comparison of an exemplary transducer probe with a conventional transducer probe, in accordance with aspects of the present specification;

FIG. 4 is a diagrammatical representation of a first interposer coupled to an electrical standoff, in accordance with aspects of the present specification;

FIG. 5 is a diagrammatical representation of a first interposer coupled to an electrical standoff and having a flex interconnect positioned between the electrical standoff and ASIC bumps, in accordance with aspects of the present specification;

FIG. 6 is a diagrammatical representation of a second interposer coupled to an electrical standoff, in accordance with aspects of the present specification;

FIG. 7 is a diagrammatical representation of a portion of an exemplary transducer probe having an electrical standoff, in accordance with aspects of the present specification; and

FIGS. 8-10 are diagrammatical representations of different embodiments of an electrical standoff, in accordance with aspects of the present specification.

### DETAILED DESCRIPTION

As will be described in detail hereinafter, various embodiments of an ultrasound transducer probe for reducing a footprint of the ultrasound transducer probe are presented. In particular, the ultrasound transducer probe and methods presented herein employ an electrical standoff to reduce the footprint of the ultrasound transducer probe. In addition to reducing the footprint of the transducer probe, the electrical standoff may also be used to attenuate ultrasound signals emitted from an acoustic array towards a flex interconnect disposed in the transducer probe. Further, the electrical standoff may be used to manage heat generated in the ultrasound transducer probe.

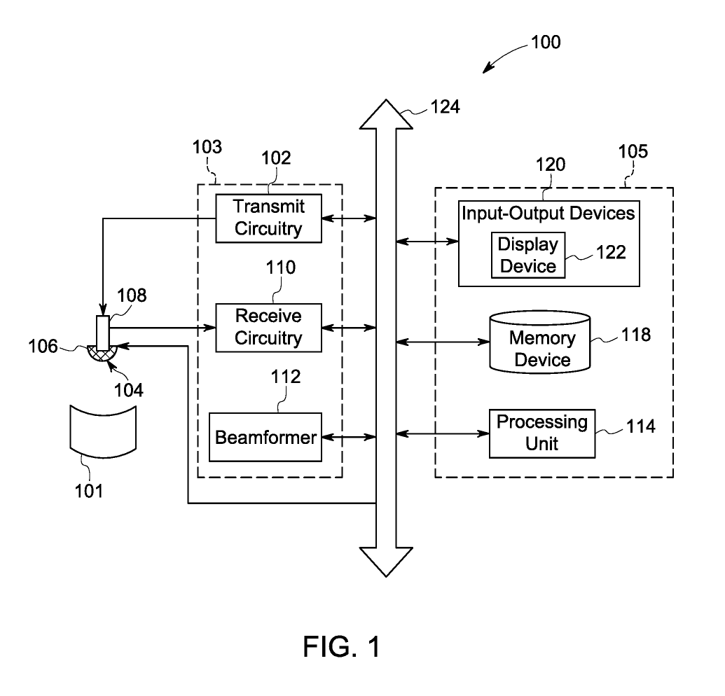

Although the following description includes embodiments relating to ultrasound imaging, these embodiments may also be implemented in other medical imaging systems that employ devices such as ultrasound and/or interventional probes during imaging. These systems, for example, may include magnetic resonance imaging (MRI) systems, computed-tomography (CT) systems, and systems that monitor targeted drug and gene delivery. Further, these medical imaging systems may be used for accurate diagnosis and staging of coronary artery disease and monitoring of therapies including high-intensity focused ultrasound (HIFU), radiofrequency ablation (RFA), and brachytherapy. An exemplary environment that is suitable for practising various implementations of the present system is described in the following sections with reference to FIG. 1.

FIG. 1 illustrates an ultrasound system 100 for imaging a target volume 101 in biological tissues of interest in a subject. In one example, the target volume 101 may include cardiac tissues, liver tissues, breast tissues, prostate tissues, thyroid tissues, lymph nodes, vascular structures adipose tissue, muscular tissue, and/or blood cells. Alternatively, the system 100 may be employed for imaging non-biological materials such as manufactured parts, plastics, aerospace composites, and/or foreign objects within a body such as a catheter or a needle. In one embodiment, the ultrasound system 100 may be a console system or a cart-based system. Alternatively, the ultrasound system 100 may be a portable system, such as a hand-held, laptop-style and/or a smartphone-based system. For ease of description, the ultrasound system 100 is represented as a portable ultrasound system.

In certain embodiments, the system 100 includes an acquisition subsystem 103 and a processing subsystem 105. The acquisition subsystem 103 is configured to obtain image data corresponding to the target volume 101. Further, the processing subsystem 105 is configured to process the acquired image data to generate one or more images corresponding to the target volume 101 in the object of interest. The acquisition subsystem 103 includes transmit circuitry 102, receive circuitry 110, and a beamformer 112. The processing subsystem 105 includes a processing unit 114, a memory device 118, and input-output devices 120.

In a presently contemplated configuration, the transmit circuitry 102 generates a pulsed waveform to drive an acoustic array 104 housed within a transducer probe 108. In accordance with embodiments of the present specification, the transducer probe 108 includes an electrical standoff to reduce a footprint of the transducer probe 108. Particularly, the pulsed waveform drives acoustic elements 106 in the acoustic array 104 to transmit ultrasonic pulses into the target volume 101. The acoustic elements 106, for example, may include piezoelectric, piezoceramic, capacitive, and/or microfabricated crystals. At least a portion of the ultrasonic pulses generated by the acoustic elements 106 is back-scattered from the target volume 101 to produce echoes that return to the acoustic array 104 and are received by receive circuitry 110 for further processing. It may be noted that the terms “ultrasonic” and “ultrasound” may be used interchangeably in the following description.

Also, in the embodiment illustrated in FIG. 1, the receive circuitry 110 is coupled to a beamformer 112 that processes the received echoes and outputs corresponding radio frequency (RF) signals. Subsequently, a processing unit 114 receives and processes the RF signals in near real-time and/or offline mode. The processing unit 114 includes devices such as one or more general-purpose or application-specific processors, digital signal processors, microcomputers, microcontrollers, application specific integrated circuits (ASICs), field programmable gate arrays (FPGA), or other suitable devices in communication with other components of the system 100. It may be noted that the various components of the ultrasound system 100 are communicatively coupled via a communication channel 124.

In addition to receiving and processing the RF signals, in certain embodiments, the processing unit 114 also provides control and timing signals for configuring one or more imaging parameters for imaging the target volume 101 in the subject. Furthermore, in one embodiment, the processing unit 114 stores the delivery sequence, frequency, time delay, and/or beam intensity, for example, in a memory device 118 for use in imaging the target volume 101. The memory device 118 includes storage devices such as a random access memory, a read only memory, a disc drive, solid-state memory device, and/or a flash memory. In one embodiment, the processing unit 114 uses the stored information for configuring the acoustic elements 106 to direct one or more groups of pulse sequences toward the target volume 101. Subsequently, the processing unit 114 tracks displacements in the target volume 101 caused in response to the incident pulses to determine corresponding tissue characteristics. The displacements and tissues characteristics, thus determined, may be stored in the memory device 118. The displacements and tissues characteristics may also be communicated to a medical practitioner, such as a radiologist, for further diagnosis.

In some embodiments, the processing unit 114 may be further coupled to one or more user input-output devices 120 for receiving commands and inputs from an operator, such as the medical practitioner. The input-output devices 120, for example, may include devices such as a keyboard, a touchscreen, a microphone, a mouse, a control panel, a display device 122, a foot switch, a hand switch, and/or a button. In one embodiment, the processing unit 114 processes the RF signal data to prepare image frames and to generate the requested medically relevant information based on user input. Particularly, the processing unit 114 may be configured to process the RF signal data to generate two-dimensional (2D) and/or three-dimensional (3D) datasets corresponding to different imaging modes.

Further, the processing unit 114 may be configured to reconstruct desired images from the 2D or 3D datasets. Subsequently, the processing unit 114 may be configured to display the desired images on the associated display device 122 that may be communicatively coupled to the processing unit 114. The display device 122, for example, may be a local device. Alternatively, in one embodiment, the display device 122 may be remotely located to allow a remotely located medical practitioner to access the reconstructed images and/or medically relevant information corresponding to the target volume 101 in the subject/patient.

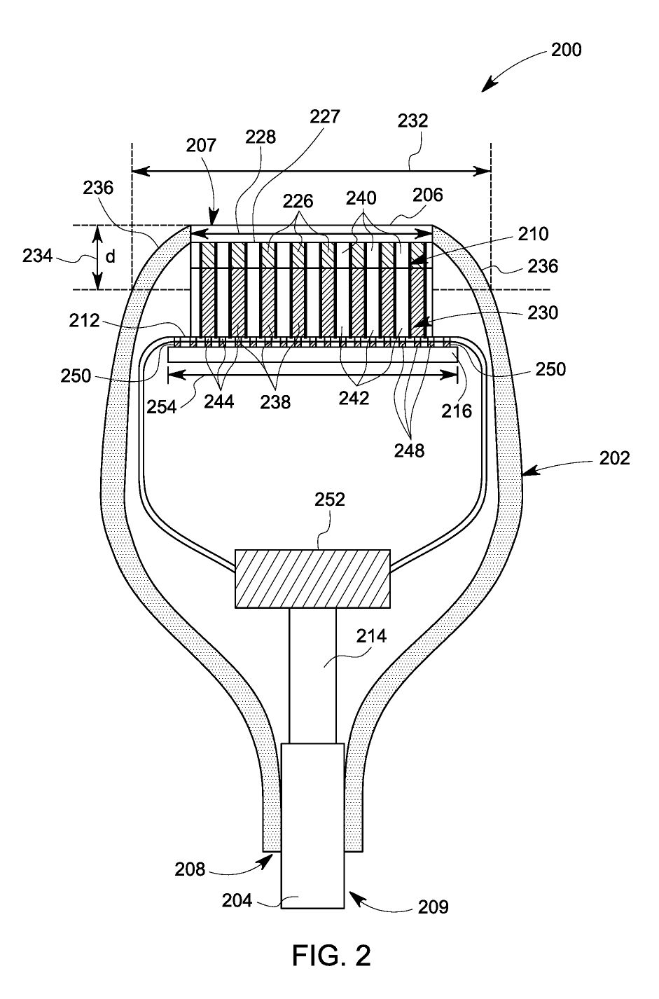

Referring to FIG. 2, a diagrammatical representation of an exemplary transducer probe 200 having an electrical standoff, in accordance with aspects of the present specification, is depicted. The transducer probe 200 is representative of one embodiment of the ultrasound transducer probe 108 of FIG. 1. The ultrasound transducer probe 200 is described with reference to the components in FIG. 1. It may be noted that the terms “ultrasound transducer probe” and “transducer probe” may be used interchangeably. The transducer probe 200 includes a housing 202 and a probe cable 204 coupled to the housing 202. The housing 202 may have one or more desired shapes depending upon the target volume 101 (see FIG. 1) in a subject/body that may be scanned using the transducer probe 200.

Further, the housing 202 may have a probe surface 206 at a first end 207 and an opening 208 at a second end 209 of the housing 202. The probe surface 206 may be a smooth closed surface that is configured to be in physical contact with the subject being scanned. In one example, the probe surface 206 may be formed using one or more materials that are used to provide mechanical protection at the first end 207 of the housing 202. In another example, the probe surface 206 may be formed using a smooth curved material that acts as a lens in the transducer probe 200.

In addition, the probe surface 206 may be configured to allow optimal positioning of the transducer probe 200 on surfaces, such as the chest, breast, and/or abdominal regions of a patient. Further, the opening 208 at the second end 209 of the housing 202 is configured to receive at least a portion of the probe cable 204 that is coupled to the housing 202, as depicted in FIG. 2. In one example, the probe surface 206 may have sides 236 that may be configured to act as sides of the housing 202.

In addition to the housing 202 and the probe cable 204, the transducer probe 200 includes an acoustic array 210, a flex interconnect 212, a sub-cable 214, and one or more application specific integrated circuit (ASIC) 216. It may be noted that the transducer probe 200 may include other components, and is not limited to the components shown in FIG. 2. The acoustic array 210 is disposed adjacent the probe surface 206 of the housing 202. In the embodiment of FIG. 2, the acoustic array 210 includes a plurality of acoustic elements 226, where adjacently disposed acoustic elements 226 of the plurality of acoustic elements 226 are separated by a gap 240.

Further, these acoustic elements 226 are arranged to form an array aperture 227 adjacent the probe surface 206 of the transducer probe 200. The array aperture 227 may have a lateral size 228. In one example, the lateral size 228 of the array aperture 227 is same as a lateral size of the acoustic array 210. Also, these acoustic elements 226 are used to transmit ultrasound signals towards the target volume 101 in the subject via the array aperture 227. In one example, the acoustic elements 226 may include a piezoelectric layer that is driven by electrical pulses to transmit the ultrasound signals towards the target volume 101. It may be noted that the number of acoustic elements 226 included in the acoustic array 210 may vary depending upon the transducer design and/or type of imaging that is to be performed.

Further, the flex interconnect 212 may include interconnects that are flexible and adaptable to provide electrical connection between the acoustic array 210, the ASIC 216, and one or more electronic units 252 in the probe 200. Further, the one or more electronic units 252 may be electrically coupled to the probe cable 204 via the sub-cable 214. The flex interconnect 212 is configured to electrically couple the acoustic array 210 to the electronic units 252. In one example, the flex interconnect 212 may be used to communicate the ultrasonic/electrical pulses between the piezoelectric layer in the acoustic elements 226 and the electronic units 252 in the probe 200. In one embodiment, the electronic units 252 may be positioned outside the transducer probe 200.

Further, the ASIC 216 is positioned adjacent the flex interconnect 212 to receive one or more ultrasound signals that are reflected from the target volume 101 in the subject. Also, the ASIC 216 may process and communicate these reflected ultrasound signals to the electronic units 252.

In addition, the ASIC 216 may include one or more input-output (I/O) connections 250 disposed along a periphery of the ASIC 216, as depicted in FIG. 2. In one example, the I/O connections 250 are routed along the flex interconnect 212. In one embodiment, these I/O connections 250 may be used for electrically coupling the ASIC 216 to the processing unit 114 for communicating the ultrasound signals to the processing unit 114. Also, due to the presence of the I/O connections 250 at the periphery of the ASIC 216, a lateral size 254 of the ASIC 216 and the flex interconnect 212 are extended beyond the lateral size 228 of the array aperture 227. Further, it is desirable for the flex interconnect 212 to maintain a minimum bending radius to avoid failures due to trace breakage of the flex interconnect 212. Thus, it is desirable for the housing 202 of the transducer probe 200 to extend such that the housing 202 covers these components of the transducer probe 200.

Existing probes may include a stacked structure consisting of an acoustic array, an interconnect, and and one or more ASICs. Also, this stacked structure is positioned adjacent a probe surface of the transducer probe. Further, a lateral size of the ASICs and a lateral size of the interconnect are substantially larger than a lateral size of the acoustic stack in the transducer probe. As a result, a footprint of the transducer probe extends beyond the lateral size of the acoustic stack. As will be appreciated, a transducer probe with such a large footprint is particularly undesirable in applications where the transducer probe needs to be maneuvered in relatively small spaces.

In accordance with aspects of the present specification, the exemplary transducer probe 200 is configured to overcome the above shortcomings. In particular, the exemplary transducer probe 200 may employ an electrical standoff 230 to reduce a footprint 232 of the transducer probe 200 to a first value. In one example, the first value is proximate to a lateral size 228 of the array aperture 227. The footprint 232 may be representative of an outer width of the transducer probe 200 at a determined depth 234 from the probe surface 206. In one example, the determined depth 234 is in a range from about 1 mm to about 4 mm.

As depicted in FIG. 2, the electrical standoff 230 may be positioned between the flex interconnect 212 and the acoustic array 210 to distance the acoustic array 210 from the flex interconnect 212. In one example, by positioning the electrical standoff 230 between the flex interconnect 212 and the acoustic array 210, the flex interconnect 212 may be positioned away from the probe surface 206 and the acoustic array 210. This in turn provides spacing within the housing 202 and allows the sides 236 of the probe surface 206 to be positioned closer to the acoustic array 210. As one or more of the sides 236 of the probe surface 206 are positioned closer to the acoustic array 210, the footprint 232 of the transducer probe 200 is reduced to the first value. In one example, the first value may relatively closely match with the lateral size 228 or be proximate to the lateral size 228 of the array aperture 227. In a non-limiting example, the footprint 232 of the exemplary transducer probe 200 is in a range from about 23 mm to about 35 mm. Advantageously, the footprint 232 of the transducer probe 200 is reduced without minimizing the array aperture 227 of the acoustic array 210.

In the embodiment of FIG. 2, the electrical standoff 230 may include a plurality of standoff elements 238, where adjacently disposed standoff elements 238 of the plurality of standoff elements are separated by corresponding gaps 242. In one embodiment, the gaps 242 may be vertical gaps and/or slanted gaps. Further, the gaps 242 may be of any desired shape. Advantageously, the slanted gaps between the standoff elements 238 may aid in improving an acoustic performance of the probe 200. By way of example, the slanted gaps between the standoff elements 238 may cause the ultrasound signals that travel in the electrical standoff 230 to encounter more interfaces, which in turn increases propagation distance and absorption of the ultrasound signals in the electrical standoff 230. In one example, these ultrasound signals may be undesirable acoustic signals that are transmitted by the acoustic array 210 towards the flex interconnect 212 and the ASIC 216.

Further, the standoff elements 238 may be fabricated from one or more electrical and thermal conductors. In one embodiment, the gaps 242 may be filled with one or more electrical insulators to isolate the standoff elements 238 from one another. Also, each of these standoff elements 238 may be aligned with a corresponding acoustic element in the acoustic array 210. In one example, each of the standoff elements 238 may be electrically coupled to at least one of the acoustic elements 226 in the acoustic array 210.

Furthermore, the flex interconnect 212 may include a plurality of pass-through connections 244. The pass-through connections 244 are used for electrically coupling the one or more standoff elements 238 in the electrical standoff 230 to the ASIC 216. In one embodiment, each of these pass-through connections 244 may be aligned with a corresponding standoff element 238 in the electrical standoff 230. In one embodiment, the ASIC 216 may include one or more ASIC bumps 248 that are used to electrically couple the ASIC 216 to the standoff elements 238 via the pass-through connections 244. Thus, the ASIC 216 is electrically coupled to the acoustic elements 226 in the acoustic array 210 through the ASIC bumps 248, the pass-through connections 244, and the standoff elements 238.

In an exemplary embodiment, the electrical standoff 230 may be used to attenuate at least a portion of the ultrasound signals transmitted towards the flex interconnect 212. Particularly, while transmitting the ultrasound signals towards the target volume, the acoustic elements 226 may transmit a portion of the ultrasound signals towards the flex interconnect 212 and the ASIC 216 in the ultrasound probe 200. These ultrasound signals may cause spurious reflections with the ultrasound signals received from the target volume 101. As a result, image artifacts may be obtained in an ultrasound image of the target volume 101. To avoid these problems, the standoff elements 238 in the electrical standoff 230 are used to absorb the ultrasound signals transmitted by the acoustic elements 226 towards the flex interconnect 212. This in turn prevents spurious reflections in the transducer probe 200.

In addition, the electrical standoff 230 may be used to manage heat generated in the ultrasound probe 200. In one example, the ASIC 216 may generate heat while processing the ultrasound signals received from the target volume 101. However, this heat may propagate towards the probe surface/lens 206 and may cause the probe 200 to overheat, which in turn may deactivate/shutdown the probe 200. To avoid this problem, the standoff elements 238 may include one or more low thermal conductive materials to thermally isolate the lens/probe surface 206 from the ASIC 216, which in turn prevents heating of the lens/probe surface 206. In another embodiment, the electrical standoff 230 may be used to remove heat generated by the acoustic array 210 and the lens/probe surface 206. Particularly, the electrical standoff 230 may provide a thermal path for the heat generated by the acoustic array 210 to propagate towards the ASIC 216, or any other thermal management components in the transducer probe 200.

Also, in one embodiment, the electrical standoff 230 may include a low parasitic capacitance that allows for reduced power consumption during transmission of the ultrasonic/electrical pulses to the acoustic elements 226 in the acoustic array 210. Further, this low parasitic capacitance may reduce noise while receiving the ultrasound signals from the target volume 101.

Thus, by employing the electrical standoff 230 in the ultrasound transducer probe 200, the footprint 232 of the transducer probe 200 is reduced to more closely match with the array aperture 227 of the acoustic array 210. Also, the footprint 232 of the transducer probe 200 is reduced without minimizing the array aperture 227 of the acoustic array 210. Furthermore, the electrical standoff 230 may aid in attenuating the ultrasound signals transmitted by the acoustic array 210, which in turn reduces spurious reflections in the ultrasound probe 200. In addition, the electrical standoff 230 may aid in managing the heat generated in the ultrasound probe 200.

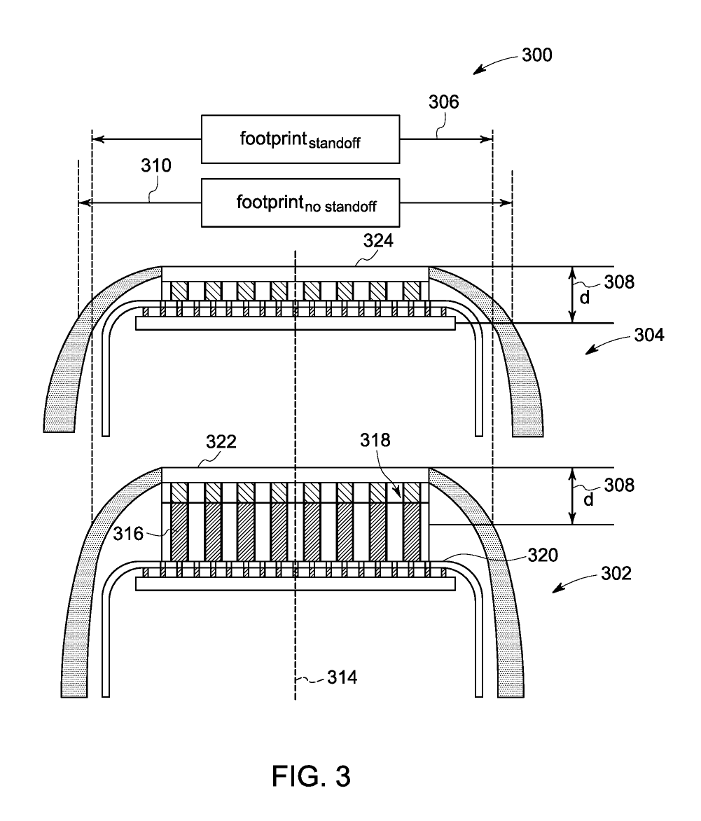

FIG. 3 is a diagrammatical representation 300 of a comparison of a portion of an exemplary transducer probe 302 with a portion of a conventional transducer probe 304. The exemplary transducer probe 302 is similar to the transducer probe 200 of FIG. 2. Reference numeral 306 represents a footprint of the transducer probe 302. The footprint 306 is determined at a depth 308 from a probe surface 322. Reference numeral 310 represents a footprint of the transducer probe 304 at the same depth 308 from a probe surface 324.

Further, the exemplary transducer probe 302 and the conventional transducer probe 304 are illustrated at a same scale and are symmetrically positioned along a vertical axis 314, as depicted in FIG. 3. By positioning an electrical standoff 316 between an acoustic array 318 and a flex interconnect 320 in the exemplary transducer probe 302, the acoustic array 318 may be distanced from the flex interconnect 320. This in turn provides spacing within a housing of the transducer probe 302 and allows sides of the probe surface 322 to be positioned closer to the acoustic array 318. As a result, the footprint 306 of the exemplary transducer probe 302 is reduced to a size lower than that of the footprint 310 of the conventional transducer probe 304, as depicted in FIG. 3. In one example, the footprint 306 of the exemplary transducer probe 302 may be reduced by about 4 mm as compared to the footprint 310 of the conventional transducer probe 304. Also, the footprint 306 may more closely match with an array aperture of the acoustic array 318, thereby enabling the probe 302 to be maneuvered in smaller spaces.

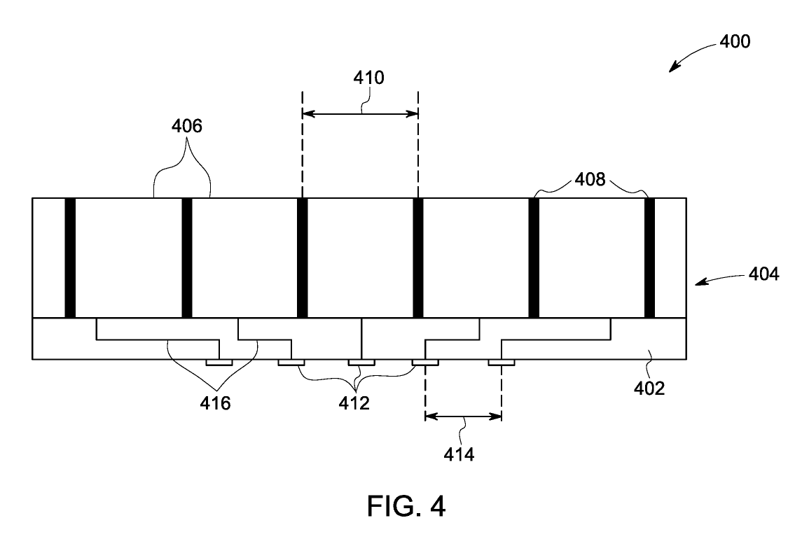

Referring to FIG. 4, a diagrammatical representation 400 of a first interposer 402 coupled to an electrical standoff 404, in accordance with aspects of the present specification, is depicted. The electrical standoff 404 is similar to the electrical standoff 230 of FIG. 2. In particular, the electrical standoff 404 includes one or more standoff elements 406 that are separated from one another by insulators 408. In one example, the standoff elements 406 may include one or more conductive materials, while the insulators 408 may include one or more insulating materials. Further, the electrical standoff 404 may have a first pitch 410. As used herein, the term “pitch” refers to a distance from the center of one element to the center of an adjacent element. In one example, these elements may be insulators in the electrical standoff, ASIC bumps in an ASIC, acoustic elements in an acoustic array, or pass-through connections in a flex interconnect. As illustrated in FIG. 4, the first pitch 410 may be representative of a distance from the center of one spacing/insulator 408 to the center of an adjacent spacing/insulator 408 in the electrical standoff 404.

In a similar manner, ASIC bumps 412 that are coupled to an ASIC (shown in FIG. 2) may have a second pitch 414. The second pitch 414 may be representative of a distance from the center of one ASIC bump 412 to the center of an adjacent ASIC bump 412. In some embodiments, the second pitch 414 of the ASIC bumps 412 may be different from the first pitch 410 of the electrical standoff 404. In one example, if the second pitch 414 of the ASIC bumps 412 is ‘x’ then the first pitch 410 of the electrical standoff 404 is ‘x+y,’ where ‘y’ is additional distance/pitch. As a result, each of the ASIC bumps 412 may not be electrically coupled to a corresponding standoff element 406 in the electrical standoff 404 when the electrical standoff 404 is positioned in the transducer probe. This in turn may affect an ultrasound imaging of a target volume in a subject.

In certain embodiments, the first interposer 402 is positioned between the electrical standoff 404 and the ASIC bumps 412 to facilitate coupling between the electrical standoff 404 and the ASIC bumps 412. In particular, the first interposer 402 is used to electrically couple the electrical standoff 404 having the first pitch 410 to the ASIC bumps 412 having the second pitch 414. In one example, the first interposer 402 may include a plurality of electrical lines 416 that facilitate connecting each of the ASIC bumps 412 with a corresponding standoff element 406 in the electrical standoff 404, as depicted in FIG. 4. In one embodiment, the electrical standoff 404 may have slanted gaps between the standoff elements 406. In one example, these slanted gaps may be filled with insulators, such as tungsten-epoxy composites to improve attenuation of the ultrasound signals. Further, the slanted gaps between the standoff elements 406 may change a pitch of the electrical standoff 404. Even in this embodiment, the first interposer 402 may be coupled/integrated with the electrical standoff 404 to facilitate electrical connection between the standoff elements 406 having the slanted gaps and the ASIC bumps 412. Thus, by employing the first interposer 402, the electrical standoff 404 may be electrically coupled to the ASIC bumps 412 even if the electrical standoff 404 and the ASIC bumps 412 have different pitches. In the embodiment of FIG. 4, a flex interconnect (see flex interconnect 212 of FIG. 2) may not be used between the electrical standoff 404 and the ASIC bumps 412. Particularly, the electrical standoff 404 may be coupled to the ASIC bumps 412 without using the flex interconnect.

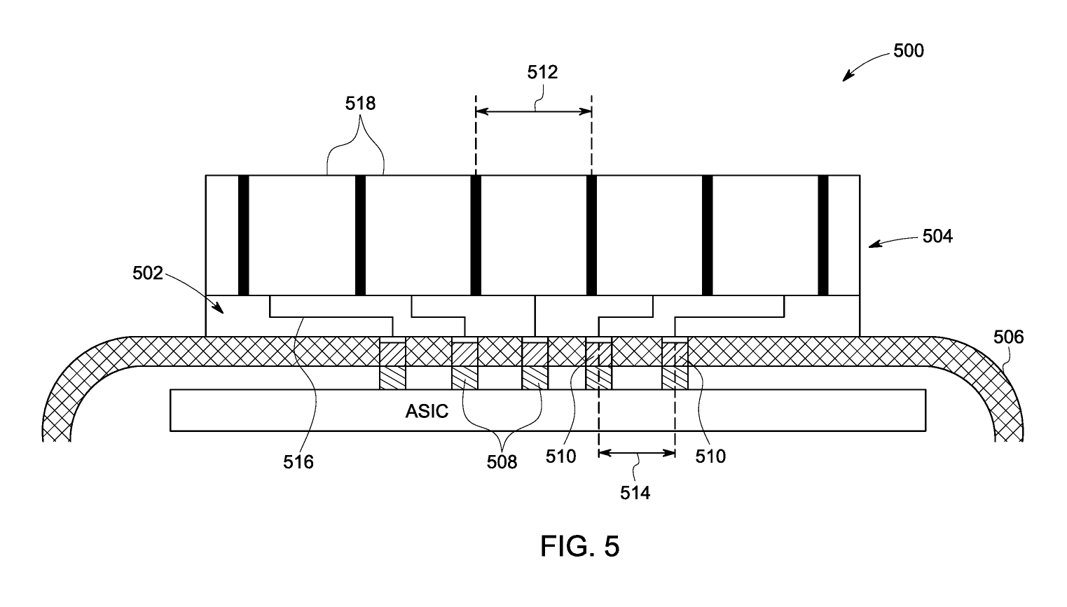

Referring now to FIG. 5, a diagrammatical representation 500 of a first interposer 502 coupled to an electrical standoff 504, in accordance with one embodiment of the present specification, is depicted. The embodiment of FIG. 5 is similar to the embodiment of FIG. 4 except that a flex interconnect 506 is positioned between the electrical standoff 504 and ASIC bumps 508. Further, the first interposer 502 is positioned between the electrical standoff 504 and the flex interconnect 506. The flex interconnect 506 is similar to the flex interconnect 212 of FIG. 1. Also, the first interposer 502 is similar to the first interposer 402 of FIG. 4.

As depicted in FIG. 5, the ASIC bumps 508 may be coupled to pass-through connections 510 of the flex interconnect 506. Further, the electrical standoff 504 may have a first pitch 512, while the flex interconnect 506 may have a second pitch 514 that is different from the first pitch 512 of the electrical standoff 504. In one example, the first pitch 512 may be similar to the first pitch 410 of FIG. 4. Further, the second pitch 514 of the flex interconnect 506 may be representative of a distance from the center of one pass-through connection 510 to the center of an adjacent pass-through connection 510 of the flex interconnect 506.

In certain embodiments, the first interposer 502 is used to electrically couple the electrical standoff 504 having the first pitch 512 to the pass-through connections 510 of the flex interconnect 506 having the second pitch 514. In one example, the first interposer 502 may include a plurality of electrical lines 516 that facilitate connecting each of the pass-through connections 510 with a corresponding standoff element 518 of the electrical standoff 504, as depicted in FIG. 5. Thus, by employing the first interposer 502, the electrical standoff 504 may be electrically coupled to the flex interconnect 506 even if the electrical standoff 504 and the flex interconnect 506 have different pitches.

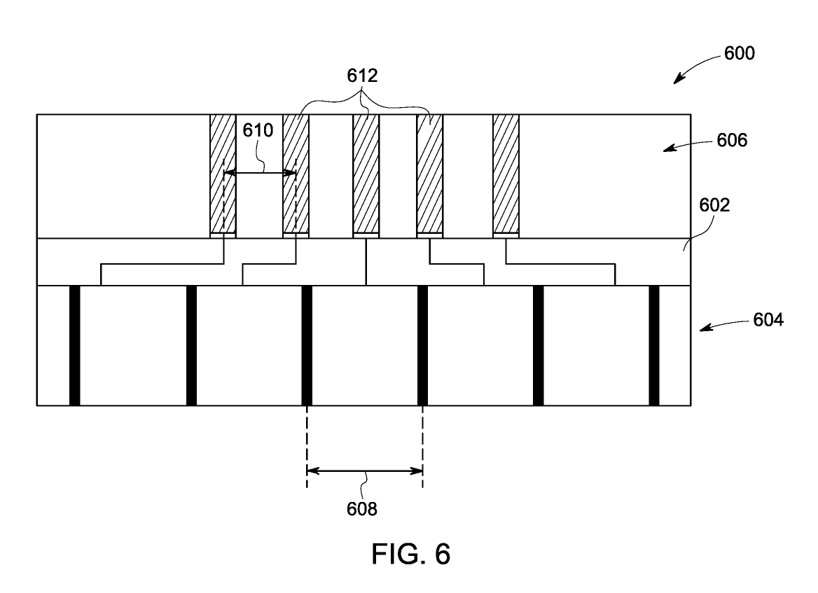

Referring now to FIG. 6, a diagrammatical representation 600 of a second interposer 602 coupled to an electrical standoff 604, in accordance with aspects of the present specification, is depicted. The second interposer 602 is similar to the first interposer 402 of FIG. 4. However, the second interposer 602 is positioned between an electrical standoff 604 and an acoustic array 606. Also, the second interposer 602 is used to electrically couple the electrical standoff 604 having a first pitch 608 to the acoustic array 606 having a second pitch 610. The first pitch 608 of the electrical standoff 604 is different from the second pitch 610 of the acoustic array 606. Also, in one example, the second pitch 610 of the acoustic array 606 may be representative of a distance from the center of one acoustic element 612 to the center of an adjacent acoustic element 612 in the acoustic array 606.

Advantageously, as illustrated in the embodiment of FIG. 6, the second interposer 602 is used to electrically couple the electrical standoff 604 having the first pitch 608 to the acoustic array 606 having the second pitch 610, where the first pitch 608 is different from the second pitch 610.

Although not illustrated, various other embodiments are envisioned. By way of example, in one embodiment, the first interposer may be positioned between the electrical standoff and the flex interconnect or the ASIC bumps. Further, in the same embodiment, the second interposer may be positioned between the electrical standoff and the acoustic array.

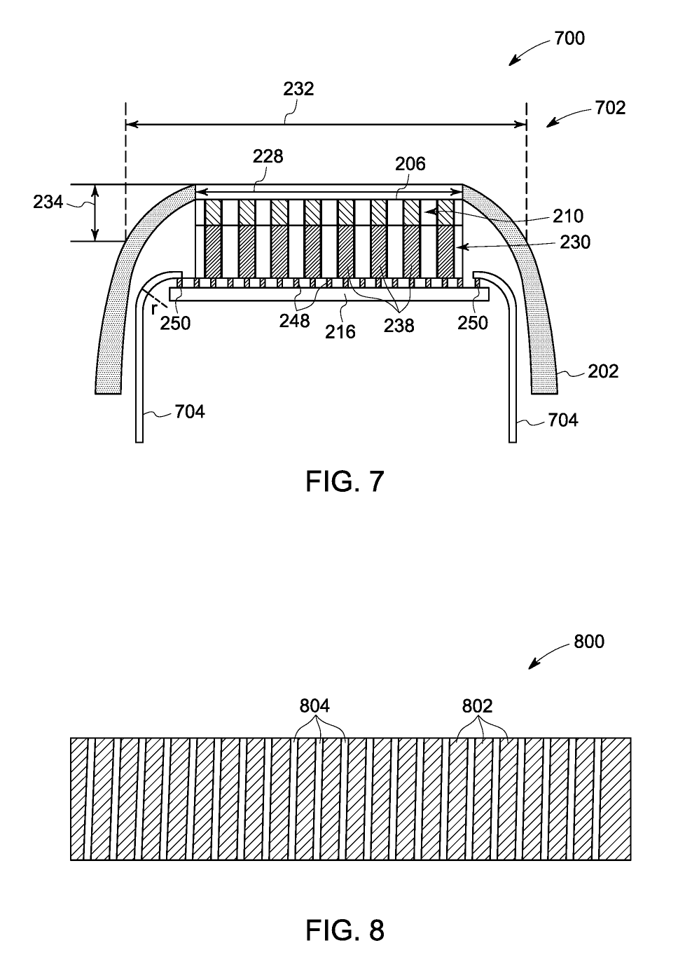

Referring to FIG. 7, a diagrammatical representation 700 of a portion of an exemplary transducer probe having an electrical standoff, in accordance with one embodiment of the present specification, is depicted. Reference numeral 702 represents a transducer probe, while reference numeral 704 represents a flex interconnect. The transducer probe 702 is similar to the transducer probe 200 of FIG. 2. However, in the transducer probe 702, the flex interconnect 704 is coupled only to input-output (I/O) connections 250 on the ASIC 216. Particularly, in the example of FIG. 7, the flex interconnect 704 is not positioned between the ASIC bumps 248 and the electrical standoff 230. Consequently, the standoff elements 238 of the electrical standoff 230 may be directly coupled to the ASIC bumps 248 without using pass-through connections of the flex interconnect 704.

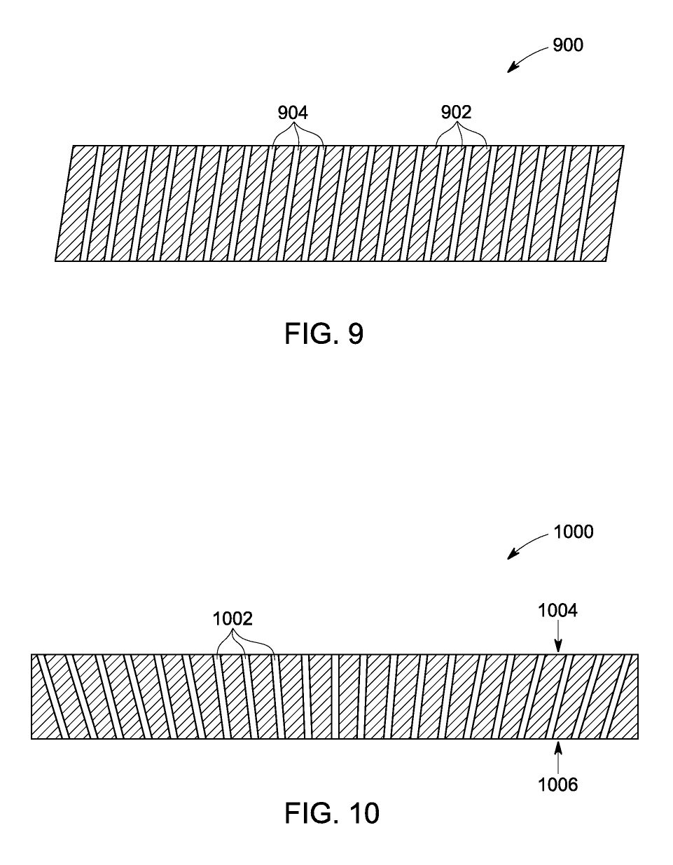

Referring now to FIGS. 8-10, diagrammatical representation of different embodiments of an electrical standoff, in accordance with aspects of the present specification, is depicted. It may be noted that the electrical standoff depicted in FIGS. 8-10 may be similar to the electrical standoff 230 of FIG. 2.

In the embodiment of FIG. 8, an electrical standoff 800 includes standoff elements 802 that are separated by vertical gaps 804. In one example, the vertical gaps 804 may be filled with one or more insulators. Also, the standoff elements 802 may act as straight conductors to communicate signals from one or more ASICs to an acoustic array (see FIG. 2).

In the embodiment of FIG. 9, an electrical standoff 900 includes standoff elements 902 that are separated by slanted gaps 904. In one example, the slanted gaps 904 may be filled with one or more insulators. Also, the standoff elements 902 may act as slanted conductors in the electrical standoff 900 to disrupt ultrasound signals that are transmitted by an acoustic array (see FIG. 2) towards a flex interconnect and/or an ASIC. Further, the disrupted ultrasound signals may scatter in the electrical standoff 900, which in turn result in improved attenuation of the ultrasound signals in a probe. Also, in one embodiment, the slanted gaps 904 between the standoff elements 902 may cause the ultrasound signals to encounter more interfaces, which in turn increases propagation distance and absorption of the ultrasound signals in the electrical standoff 900. As a result, attenuation of the ultrasound signals may be improved in the probe.

Further, in the embodiment of FIG. 10, an electrical standoff 1000 includes standoff elements 1002 having an integrated redistribution structure. It may be noted that the integrated redistribution structure may be representative of a structure, where one or more of the standoff elements 1002 may converge with respect to one another from a first end 1004 of the electrical standoff 1000 to a second end 1006 of the electrical standoff 1000, as depicted in FIG. 10. As a result of this integrated redistribution structure, the electrical standoff 1000 may have a first pitch at the first end 1004 and a second pitch at the second end 1006 of the electrical standoff 1000, where the first pitch is different from the second pitch. This change in pitches at the first end 1004 and the second end 1006 of the electrical standoff 1000 may aid in electrically coupling the acoustic array to the flex interconnect/ASIC bumps even if the acoustic array and the flex interconnect/ASIC bumps have different pitches. Advantageously, this integrated redistribution structure of the electrical standoff 1000 may facilitate electrical connection between the acoustic stack and the flex interconnect/ASIC without using an interposer. Furthermore, in one embodiment, the electrical standoffs 800, 900, 1000 depicted in FIGS. 8-10 may be printed by using a three dimensional (3D) printer.

The various embodiments of the exemplary system aid in reducing the footprint of the transducer probe without minimizing the array aperture of the acoustic array. Also, the footprint of the transducer probe is more closely matched with the array aperture of the acoustic array. In addition, the ultrasound signals transmitted by the acoustic array towards the flex interconnect are attenuated to minimize spurious reflections in the transducer probe. Moreover, the heat generated in the transducer probe may be conducted away from the lens/probe surface, or prevented from being conducted towards the lens/probe surface, which in turn prevents the acoustic array and the lens/probe surface from overheating during operation of the ultrasound probe.

While only certain features of the present disclosure have been illustrated and described herein, many modifications and changes will occur to those skilled in the art. It is, therefore, to be understood that the appended claims are intended to cover all such modifications and changes as fall within the true spirit of the present disclosure.

1. A transducer probe, comprising:
a housing having a probe surface at a first end;
an acoustic array having an array aperture, wherein the acoustic array is disposed adjacent the probe surface of the housing, and wherein the acoustic array is configured to transmit ultrasound signals towards a target volume;
a flex interconnect configured to electrically couple the acoustic array to at least one electronic unit; and
an electrical standoff disposed between the acoustic array and the flex interconnect to reduce a footprint of the transducer probe to a first value, wherein the first value is proximate to a lateral size of the array aperture.

2. The transducer probe of claim 1, wherein the electrical standoff is configured to distance the acoustic array from the flex interconnect to reduce the footprint of the transducer probe.

3. The transducer probe of claim 1, wherein the acoustic array comprises a plurality of acoustic elements, and wherein adjacently disposed acoustic elements of the plurality of acoustic elements are separated by a gap.

4. The transducer probe of claim 3, wherein each of the plurality of standoff elements is electrically coupled to at least one acoustic element of the plurality of acoustic elements.

5. The transducer probe of claim 4, wherein the flex interconnect comprises a plurality of pass-through connections, and wherein each pass-through connection of the plurality of pass-through connections is aligned with a corresponding standoff element of the plurality of standoff elements.

6. The transducer probe of claim 5, further comprising at least one application specific integrated circuit (ASIC) electrically coupled to one or more of the plurality of standoff elements and configured to process the ultrasound signals received from the target volume.

7. The transducer probe of claim 6, wherein the at least one ASIC comprises a plurality of ASIC bumps configured to electrically couple at least a portion of the ASIC to the plurality of standoff elements via the pass-through connections in the flex interconnect.

8. The transducer probe of claim 6, further comprising one or more input-output (I/O) connections disposed along a periphery of the ASIC, wherein the one or more input-output connections are routed along a length of the flex interconnect.

9. The transducer probe of claim 1, further comprising a first interposer disposed between the electrical standoff and the flex interconnect and configured to electrically couple the electrical standoff to the flex interconnect, and wherein a pitch of the electrical standoff is different from a pitch of the flex interconnect.

10. The transducer probe of claim 1, further comprising a second interposer disposed between the electrical standoff and the acoustic array and configured to electrically couple the electrical standoff to the acoustic array, wherein a pitch of the electrical standoff is different from a pitch of the acoustic array.

11. The transducer probe of claim 1, wherein the electrical standoff comprises an integrated redistribution structure configured to electrically couple the acoustic array to the flex interconnect, and wherein a pitch of the acoustic array is different from a pitch of the flex interconnect.

12. A system for ultrasound imaging, the system comprising:
an acquisition subsystem configured to obtain image data corresponding to a target volume in an object of interest and comprising an ultrasound probe, wherein the ultrasound probe comprises:
a housing having a first end and a second end, wherein the first end comprises a probe surface, and wherein the second end is coupled to a probe cable;
an acoustic array having an array aperture, wherein the acoustic array is disposed adjacent the probe surface of the housing, and wherein the acoustic array is configured to transmit ultrasound signals towards the target volume;
a flex interconnect configured to electrically couple the acoustic array to at least one electronic unit;
an electrical standoff disposed between the acoustic array and the flex interconnect to reduce a footprint of the transducer probe to a first value, wherein the first value is proximate to a lateral size of the array aperture; and
a processing subsystem in operative association with the acquisition subsystem and configured to process the acquired image data to generate one or more images corresponding to the target volume in the object of interest.

13. The system of claim 12, wherein the electrical standoff is configured to distance the acoustic array from the flex interconnect to reduce the footprint of the transducer probe.

14. A transducer probe, comprising:
a housing having a probe surface at a first end;
an acoustic array having an array aperture, wherein the acoustic array is disposed adjacent the probe surface of the housing, and wherein the acoustic array is configured to transmit ultrasound signals towards a target volume;
at least one ASIC configured to electrically couple the acoustic array and configured to receive the ultrasound signals reflected from the target volume; and
an electrical standoff disposed between the acoustic array and the at least one ASIC to reduce a footprint of the transducer probe to a first value, wherein the first value is proximate to a lateral size of the array.

15. The transducer probe of claim 14, wherein the electrical standoff is configured to distance the acoustic array from the at least one ASIC to reduce the footprint of the transducer probe.

16. The transducer probe of claim 14, wherein the at least one ASIC comprises at least one input-output connection disposed along a periphery of the at least one ASIC.

17. The transducer probe of claim 16, further comprising a flex interconnect coupled to the at least one input-output connection and configured to electrically couple the ASIC to one or more electronic units in the transducer probe.

18. The transducer probe of claim 14, wherein the at least one ASIC comprises a plurality of ASIC bumps configured to electrically couple the at least one ASIC to a plurality of standoff elements in the electrical standoff.

19. The transducer probe of claim 14, further comprising a first interposer configured to electrically couple the electrical stand off and the ASIC, wherein a pitch of the electrical standoff is different from a pitch of the ASIC.

20. The transducer probe of claim 14, wherein the electrical standoff comprises an integrated redistribution structure configured to electrically couple the acoustic array to the ASIC, and wherein a pitch of the acoustic array is different from a pitch of the ASIC.
